(19) United States
(12) Patent Application Publication
(10) Pub. No.: US 2022/0280422 A1
(43) Pub. Date: Sep. 8, 2022

(54) SOLUBILIZED MELATONIN

(71) Applicant: Innovation for Success, LLC, Doylestown, PA (US)

(72) Inventor: Leonard W. Heflich, Doylestown, PA (US)

(21) Appl. No.: 17/650,156

(22) Filed: Feb. 7, 2022

Related U.S. Application Data
(60) Provisional application No. 63/156,406, filed on Mar. 4, 2021.

(51) Int. Cl. A61K 9/00; A61K 9/127; A61K 31/4045

(52) U.S. Cl. CPC A61K 9/0056; A61K 9/1277; A61K 31/4045

(57) ABSTRACT

A method of making an orally-dissolvable strip includes combining melatonin with glycerin, forming a melatonin solution by heating the combined melatonin and glycerin to dissolve the melatonin in the glycerin, and forming a plurality of liposomes by mixing a liposomal base with the melatonin solution. Each liposome of the plurality of liposomes includes a lipid shell of the liposomal base encapsulating a portion of the melatonin solution. The method further includes forming the orally-dissolvable strip by combining the plurality of liposomes with a base material. The base material and the liposomal base are orally-dissolvable. The lipid shell prevents crystallization of the dissolved melatonin in the glycerin, such that within each liposome of the plurality of liposomes the melatonin is dissolved in the glycerin.

This application claims the benefit of priority of U.S. provisional application Ser. No. 63/156,406, filed on Mar. 4, 2021 the disclosure of which is herein incorporated by reference in its entirety.

### FIELD

This disclosure relates to the field of therapeutic, nutritional, and medicinal supplements and, in particular, to optimizing delivery of supplements to the body including optimizing the delivery of melatonin to the body.

### BACKGROUND

Melatonin (N-acetyl-5-methoxy tryptamine) is believed to be the first significant antioxidant utilized by life, enabling early life forms to survive exposure to oxygen and sunlight. Effects of melatonin were first observed in 1917 by Carey Pratt McCord and Floyd P. Allen, through the lightening of the skin of tadpole tails in response to the presence of an extract of bovine pineal gland. Melatonin was discovered to be the causative factor of the lightening in 1958 by Aaron B. Lerner at Yale University, and the bovine pineal gland extract was given the name melatonin (mela—dark, tonin—lightening) due to the skin-lightening effect. Researchers have recently found that melatonin is involved in many cellular functions in both plants and animals.

Melatonin is produced in the pineal gland and released into the bloodstream to control sleep and circadian rhythm. This is the best-known function of melatonin, and many dietary supplement products are in the market, designed specifically to aid in sleep management. Melatonin, however, is also produced in mitochondria where it neutralizes highly reactive free radical molecules, especially hydroxyl free radicals, that are produced as a natural byproduct of energy production via respiration. The accumulation of damage from hydroxyl free radicals and other free radicals results in a reduction of organismal capabilities that is commonly referred to as “aging.” Due to protection from free radicals provided by melatonin, melatonin has been called the “anti-aging molecule.”

Free radical molecules are also produced by the immune system to combat pathogens, foreign materials, and other adversaries that have entered the body. In response to some adversaries, including at least some variants of the SARS-CoV-2 virus, the immune system may overact in a process referred as a cytokine storm. In some patients, the cytokine storm causes more damage than the adversary, and can even result in severe tissue damage and death. The cytokine storm that occurs in viral infections is an example of excess free radical molecules produced by the immune system causing tissue damage and death. Antioxidants applied systemically or directly to the site of production of the free radical molecules can mute the immune response and calm the cytokine storm, thereby reducing tissue damage while allowing the immune system to fight the adversary.

Moreover, researchers have found that melatonin is involved in other bodily functions, such as blood pressure regulation, the reduction of inflammation, the reproductive cycle, and protection against ultraviolet (“UV”) radiation and other forms of radiation.

Based on the above, further advancements in the administration and delivery of melatonin are desirable.

### SUMMARY

According to an exemplary embodiment of the disclosure, a method of making an orally-dissolvable strip includes combining melatonin with glycerin, forming a melatonin solution by heating the combined melatonin and glycerin to dissolve the melatonin in the glycerin, and forming a plurality of liposomes by mixing a liposomal base with the melatonin solution. Each liposome of the plurality of liposomes includes a lipid shell of the liposomal base encapsulating a portion of the melatonin solution. The method further includes forming the orally-dissolvable strip by combining the plurality of liposomes with a base material. The base material and the liposomal base are orally-dissolvable. The lipid shell prevents crystallization of the dissolved melatonin in the glycerin, such that within each liposome of the plurality of liposomes the melatonin is dissolved in the glycerin.

According to another exemplary embodiment of the disclosure an orally-dissolvable strip includes an orally-dissolvable base material, a melatonin solution, and a plurality of liposomes. The melatonin solution includes solubilized melatonin in liquid glycerin. The liposomes of the plurality of liposomes are fixedly positioned throughout the base material. Each liposome of the plurality of liposomes includes a lipid shell encapsulating a portion of the melatonin solution. The lipid shell is formed from an orally-dissolvable liposomal base. The lipid shell is configured to prevent crystallization of the melatonin of the encapsulated portion of the melatonin solution, such that each liposome of the plurality of liposomes contains the solubilized melatonin in liquid glycerin.

According to a further exemplary embodiment a topical skin care product, includes a lotion base, a melatonin solution, and a plurality of liposomes. The melatonin solution includes solubilized melatonin in liquid glycerin. The plurality of liposomes is mixed with the lotion base. Each liposome of the plurality of liposomes includes a lipid shell encapsulating a portion of the melatonin solution. The lipid shell is configured to prevent crystallization of the melatonin of the encapsulated portion of the melatonin solution, such that each liposome of the plurality of liposomes contains the solubilized melatonin in liquid glycerin.

According to yet another exemplary embodiment, the melatonin is fully solubilized to assure full bioavailability and absorption.

According to another exemplary embodiment of the disclosure, fully solubilized melatonin solution is stabilized in a liposomal matrix to preventing recrystallization of the melatonin.

According to a further exemplary embodiment of the disclosure, solubilized melatonin is incorporated into a gum-based strip that is stable, convenient, inexpensive, and effective.

According to yet another exemplary embodiment of disclosure, a method delivers melatonin directly into the bloodstream via sublingual administration, and bypasses the digestive system and liver to allow more melatonin to enter the bloodstream quickly.

The gum-based strip, disclosed herein, contains a sufficient amount of fully solubilized, stabilized melatonin. Producing a stable form of solubilized melatonin at a high enough concentration to incorporate into a tiny strip product is novel and unprecedented. This disclosure stabilizes the solubilized melatonin in a liposomal matrix that prevents recrystallization and retains the benefits of a solubilized melatonin in a convenient, dry, gum-based strip.

According to a further embodiment of the disclosure, a method is for delivering solubilized melatonin, stabilized in a liposomal matrix, directly into the lungs of a patient via nebulization, or any other inhaled dosage form, in order to apply the active, bioactive ingredient (i.e., melatonin) directly to the site of excess free radical production. The excess free radial production, in one embodiment, occurring as part of the cytokine storm caused by viral infections such as the influenza virus, the SARS-CoV-2 virus, and the like.

### BRIEF DESCRIPTION OF THE FIGURES

FIG. 1 is a top plan view of a flexible orally-dissolvable strip including solubilized melatonin, as disclosed herein;

FIG. 2 is an enlarged cross sectional view of a portion the orally-dissolvable strip taken along line II-II of FIG. 1 and showing a plurality of liposomes having a melatonin solution therein;

FIG. 3 is a flowchart illustrating an exemplary process for forming the orally-dissolvable strip of FIG. 1;

FIG. 4 is a cross-sectional view of a topical skin care product having a plurality of liposomes each encapsulating a melatonin solution including solubilized melatonin;

FIG. 5 is a flowchart illustrating an exemplary process for forming the skin care product of FIG. 4;

FIG. 6 illustrates the skin care product of FIG. 4 in cross section and applied to the skin of a patient;

FIG. 7 is a flowchart illustrating an exemplary process for forming an inhalable melatonin mixture;

FIG. 8 is a block diagram illustration of a patient utilizing a nebulization system to inhalation administer the inhalable melatonin mixture of FIG. 7; and

FIG. 9 is a block diagram illustration of a patient utilizing a vaporization system to inhalation administer the inhalable melatonin mixture of FIG. 7.

### DETAILED DESCRIPTION

For the purpose of promoting an understanding of the principles of the disclosure, reference will now be made to the embodiments illustrated in the drawings and described in the following written specification. It is understood that no limitation to the scope of the disclosure is thereby intended. It is further understood that this disclosure includes any alterations and modifications to the illustrated embodiments and includes further applications of the principles of the disclosure as would normally occur to one skilled in the art to which this disclosure pertains.

Aspects of the disclosure are disclosed in the accompanying description. Alternate embodiments of the disclosure and their equivalents may be devised without parting from the spirit or scope of the disclosure. It should be noted that any discussion herein regarding “one embodiment,” “an embodiment,” “an exemplary embodiment,” and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

For the purposes of the disclosure, the phrase “A and/or B” means (A), (B), or (A and B). For the purposes of the disclosure, the phrase “A, B, and/or C” means (A), (B), (C), (A and B), (A and C), (B and C), or (A, B and C).

The terms “comprising,” “including,” “having,” and the like, as used with respect to embodiments of the disclosure, are synonymous.

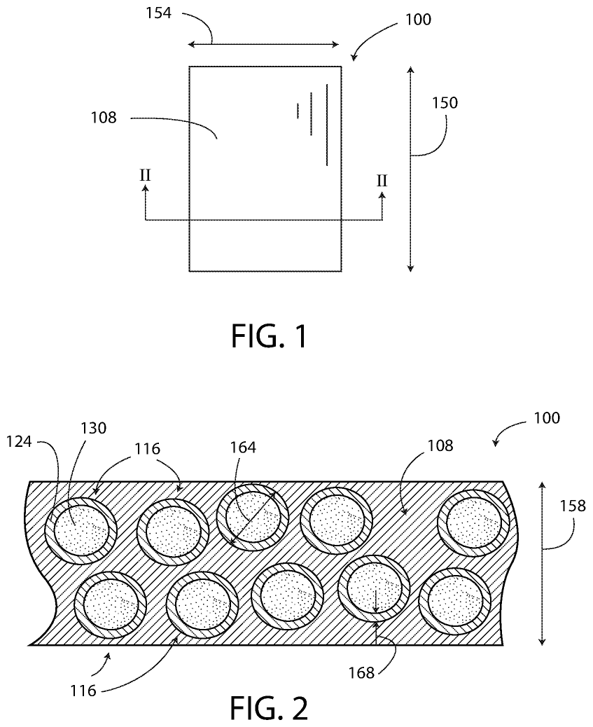

As shown in FIGS. 1 and 2, a flexible orally-dissolvable strip 100 includes a base material 108 and a plurality of liposomes 116 substantially evenly dispersed through the base material 108. The dissolvable strip 100 is also referred to herein as a dissolving film, an oral drug strip, a supplement strip, a melatonin strip, and a therapeutic dosage form. The dissolvable strip 100, which is about the size of a postage stamp (i.e., 2 cm by 3 cm) and weighs from about eighty to ninety milligrams (80 mg to 90 mg), is a convenient means of administering or delivering melatonin to a patient (human or animal) buccally, sublingually, and/or enterically. As set forth herein, the liposomes 116 include a lipid shell 124 that surrounds a highly-concentrated melatonin solution 130 that is solubilized and stabilized. The liposomes 116 prevent crystallization and/or recrystallization of the melatonin in the melatonin solution 130. The components of the strip 100 and a method 300 for producing the strip 100 are described herein.

The base material 108 is gum-based and is orally-dissolvable meaning that the base material 108 is configured to turn from a flexible film into a liquid when placed in the mouth of a patient and exposed to saliva and/or other digestive fluids. In one embodiment, the base material 108 is gum-based and is formed from or includes pullulan, which is a polysaccharide polymer that is edible, mostly tasteless, and is easily and quickly dissolvable in the mouth. The base material 108 may also include gum arabic, sodium alginate, and/or any other chemical or polymer typically used in the production of orally dissolvable thin films.

In some embodiments, the base material 108 is a “matrix” or a “gum matrix,” since the base material 108 encloses, surrounds, and protects the liposomes 116. The base material 108 is configured to protect the liposomes 116 by stabilizing the position of the liposomes 116 within the strip 100, thereby preventing destruction of the liposomes 116 from sheer forces and/or compressive forces, which could burst or otherwise break the liposomes 116. The liposomes 116 are prevented from moving within the base material 108.

As noted above, the dissolvable strip 100 is flexible. Accordingly, in some embodiments, the base material 108 includes a plasticizer in order to improve the flexibility of the strip 100 and to reduce the brittleness of strip 100. Suitable plasticizers include glycerol, propylene glycol, and others. The plasticizer also tends to reduce the likelihood of the dissolvable strips 100 from sticking together.

In an exemplary embodiment, the dissolvable strip 100 has a length 150 (FIG. 1) of about 3 cm and a width 154 (FIG. 1) of about 2 cm. The base material 108 defines an overall thickness 158 (FIG. 2) of the dissolvable strip 100. An exemplary thickness 158 of the base material 108 is less than one millimeter (1 mm) and in a specific embodiment, the base material 108 defines a thickness 158 of about 0.1 mm. In other embodiments, the dissolvable strip 100 is any desired length 150, width 154, and thickness 158 so as provide a desired predetermined dosage of the melatonin to the patient.

With reference to FIG. 2, each liposome 116 includes the lipid shell 124 surrounding or encapsulating a quantity or a portion of the melatonin solution 130. The liposomes 116 are fixedly positioned throughout the base material 108. The plurality of liposomes 116 are also referred to as a liposomal matrix and/or a liposomal dispersion. Each liposome 116 is a generally spherical vesicle as defined by the shape of the lipid shell 124.

The lipid shell 124 is formed from an orally-dissolvable liposomal base. In one embodiment, the lipid shell 124 defines at least one lipid bilayer, which is also referred to herein as a bilayer membrane of the liposome 116. A suitable lipid shell 124 that is orally-dissolvable and that defines the at least one lipid bilayer is formed from a phospholipid, such as phosphatidylcholine. In a specific embodiment, the lipid shell 124 is formed from purified soy phosphatidylcholine. In other embodiments, the lipid shell 124 is formed from any other suitable material that is configured to contain a portion of the melatonin solution 130 and that is orally-dissolvable. The lipid shell 124 is orally-dissolvable when the material forming the lipid shell 124 is configured to turn into a liquid when placed in the mouth of a patient and exposed to saliva and/or other digestive fluids. When the lipid shell 124 dissolves and/or starts to dissolve, the liposome 116 is configured to break, tear, and/or rupture thereby releasing the melatonin solution 130.

For ease of explanation, the liposomes 116 of FIG. 2 are shown very much enlarged compared to the thickness of the 158 of the base material 108. The liposomes 116 have an actual diameter 164 of about 30 nm to about 300 nm. The liposomes 116 have a corresponding lipid shell 124 thickness 168 of about 10 nm to about 30 nm. Accordingly, the dissolvable strip 100 may include from millions to billions or more of the liposomes 116. Moreover, the liposomes 116 are substantially uniformly distributed throughout the base material 108. As used herein, a substantially uniform distribution means that there is about the same number of liposomes 116 per unit of area of the dissolvable strip 100. This distribution is also referred to herein as a homogenous dispersing of the liposomes 116. An exemplary orally-dissolvable strip 100 having a substantially uniform distribution of the liposomes 116 includes approximately two million liposomes 116 per square millimeter of the strip 100.

The melatonin solution 130 is contained by the shells 124 of the liposomes 116 and includes a highly-concentrated solution of fully-solubilized melatonin. In one embodiment, the melatonin solution 130 includes melatonin fully-dissolved and fully-solubilized in liquid glycerin. Specifically, the melatonin solution 130 includes from 100 mg to 300 mg dissolved or solubilized melatonin per milliliter of glycerin. In a particular embodiment, the melatonin solution 130 includes 250 mg of melatonin per milliliter of glycerin.

Fully-dissolved and fully-solubilized melatonin, as used herein, means that there is no solid form (solid phase) melatonin in the melatonin solution 130. Typically, melatonin in the solid form is a crystalline or crystalized material. The melatonin solution 130, however, is a liquid with no crystals or other solid phase melatonin.

Melatonin is a tiny, amphipathic molecule capable of passing unimpeded through cell walls of the patient and the mitochondrial membranes of cells of the patient. The ability of melatonin to enter cells and mitochondria enables it to neutralize free radical molecules before the free radical molecules can react with protein, fat, or DNA, causing damage to the human or animal.

During research of the dissolvable strip 100, several food-grade solvents were identified having a sufficient solubility of melatonin including glycerin, ethanol, oleic acid, and propylene glycol. When heated, each of these solvents is capable of dissolving crystalized melatonin. When cooled, however, the melatonin recrystallized in each of the solvents except for the liquid glycerin, thereby making glycerin an optimal solvent for the melatonin solution 130. It was determined that ethanol is unsuitable for use in the melatonin solution 130, because the ethanol evaporates from the liposomes 116 in the strip 100 resulting in recrystallization of the melatonin. It was further determined that oleic acid does not incorporate well into the base material 108, and that propylene glycol does not produce a suitable strip product. Whereas, as noted above, glycerin incorporates well into the strip 100 at levels up to about 2% to 5%, is compatible with the lipid shell 124, and prevents recrystallization of the melatonin contained by the lipid shell 124.

When the melatonin solution 130 is contained within the lipid shell 124 of the liposome 116, the lipid shell 124 prevents crystallization and/or recrystallization of the melatonin of the melatonin solution 130. Accordingly, the melatonin of the melatonin solution 130 is stabilized and is prevented from crystalizing and/or recrystallizing in the dissolvable strip 100 and the melatonin remains dissolved in the glycerin. Specifically, the melatonin solution 130 is encapsulated by the lipid shells 124 of the liposomes 116, thereby preventing any reactions that may trigger recrystallization of the melatonin in the liquid glycerin. For example, crystallization is prevented because the glycerin of the melatonin solution 130 is almost entirely prevented from evaporating when encapsulated by the shells 124 of the liposomes 116. Stated differently, the liposomes 116 prevent crystallization of the melatonin by encapsulating a portion of the liquid melatonin solution 130, such that the melatonin is fully dissolved and/or fully solubilized in the liquid glycerin within the liposome 116.

By preventing evaporation of the glycerin using the liposomes 116, the concentration of melatonin per volume of the melatonin solution 130 remains substantially constant for at least six to twelve months, and recrystallization of the melatonin is prevented for at least six to twelve months. The melatonin solution 130 within the liposomes 116 remains a liquid even when the dissolvable strip 100 is dried and ready for sale. Using glycerin in the melatonin solution 130 was found to be an optimal solvent that suitably dissolves the melatonin and also prevents recrystallization after drying of the strip 100.

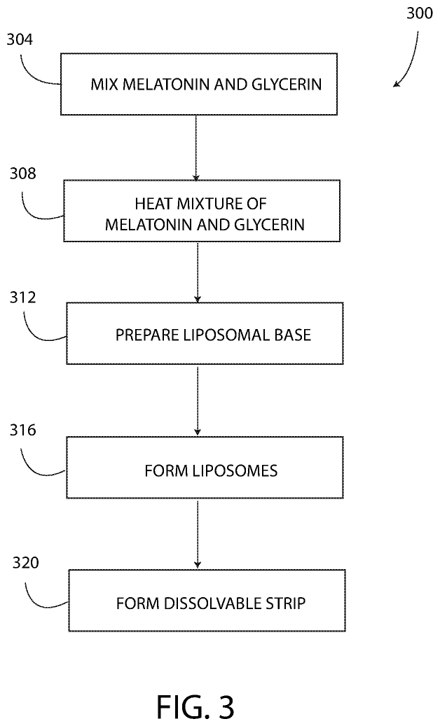

As shown in FIG. 3, an exemplary method 300 of forming the dissolvable strip 100 is set forth. At block 304, the method 300 includes combining melatonin with liquid glycerin, which is also termed glycerol and/or glycerine. The melatonin may be dry and/or powered, and is typically in a crystalized state. As noted above, in an exemplary embodiment, at least 250 mg of melatonin is added per milliliter of glycerin. When the melatonin is added to room temperature (i.e., 25° C.) glycerin, only a portion of the melatonin dissolves in the glycerin.

Next, at block 308 of the method 300, the melatonin and glycerin mixture is heated to approximately 76.6° C. (170° F.). Moreover, in some embodiments, the melatonin and glycerin mixture is stirred during the heating process. As the mixture is heated and stirred, all of the melatonin dissolves in the glycerin to form the melatonin solution 130.

At block 312 of the method 300, the liposomal base is produced with a mixture of water and purified soy phosphatidylcholine. In an exemplary embodiment, about 40 parts water are combined with 1 part soy phosphatidylcholine to form the liposomal base. The mixture is heated to 48.9° C. (120° F.) while being stirred to complete the liposomal base. In another exemplary embodiment, about 95 parts water are combined with 1 part soy phosphatidylcholine to form the liposomal base, and the mixture is heated. Accordingly, the liposomal base is formed from mixing 1 part soy phosphatidylcholine with from about 30 to 98 parts water.

At block 316, the liposomes 116 are formed. First, the melatonin solution 130 is combined with the liposomal base. In an exemplary embodiment, about 3.3 parts liposomal base is combined with 1 part of the melatonin solution 130. The combined ingredients as a percentage by weight include 75% water, 23% melatonin solution 130, and 2% soy phosphatidylcholine. In another suitable embodiment, the combined ingredients as a percentage by weight include about 95% to 98% water, about 1% melatonin solution 130, and about 1% soy phosphatidylcholine.

Next, at block 316 of the method 300, the mixture of the melatonin solution 130 and the liposomal base is sonicated while being further mixed together to form the liposomes 116. The sonication results in the liposomal base forming the lipid shells 124 around very small portions of the liquid melatonin solution 130. Any other suitable process may be used to form the liposomes 116 including, but not limited to, high-shear mixing, extrusion, micromixing, and the Mozafari method. Moreover, in one embodiment, after the liposomes 116 are formed, the mixture of the melatonin solution 130 and the liposomal base is sonicated without mixing for an additional fifteen to thirty seconds. The concentration of melatonin in the liposomes 116 is from about 1 mg/ml to about 3 mg/ml. Preferably, the concentration of melatonin in the liposomes 116 is about 1.82 mg/ml. For example, the mixture of the melatonin solution 130 and the liposomal base forms a fluid mixture that is the plurality of liposomes 116 and that can be measured by volume. A milliliter of the plurality of liposomes 116 includes, for example, 1.82 mg of melatonin.

In one embodiment, the melatonin solution 130 is maintained at a temperature of 76.6° C. until it is combined with the liposomal base. The combination of the melatonin solution 130 and the liposomal base is mixed and sonicated quickly (i.e., within five minutes and preferably within two minutes) of being combined to prevent the temperature of the melatonin solution 130 from dropping to an unsuitable level. By maintaining the temperature of the melatonin solution 130 at or near 76.6° C. prior to combining with the liposomal base, the melatonin stays fully solubilized and dissolved within the melatonin solution 130 without settling, precipitating, crystalizing, or recrystallizing. In one embodiment, the liposomes 116 are stable for a few days (i.e., three days) prior to being mixed with the base material 108. Preferably, however, the liposomes 116 are incorporated into the base material 108 shortly after production by vigorous mixing. The vigorous mixing does not damage the liposomes 116 and does not break the lipid shells 124 of the liposomes 116.

Next, at block 320 of the method 300, the orally-dissolvable strip 100 is formed. In particular, the fluid mixture of the plurality of liposomes 116 is mixed with the base material 108 to form a thin-film mixture. The thin-film mixture includes the plurality of liposomes 116 uniformly and homogeneously dispersed throughout the base material 108. The liposomes 116 are mixed with the base material 108 in a manner that prevents breaking the lipid shells 124 of the liposomes 116, such that the lipid shells 124 encapsulate the melatonin solution 130 before, during, and after the mixing with the base material 108. To prevent breaking the lipid shells 124, in one embodiment, the fluid mixture of the plurality of liposomes 116 is vigorous mixed with the base material 108 only until the uniform and homogenous mixture is achieved.

In an exemplary embodiment, the base material 108 is added to the liposomes 116 at a rate of about 2%-4% w/w and mixed until dissolved uniformly to form the thin-film mixture. The resulting thin-film mixture has a melatonin concentration of from 0.5 to 3.0 mg of melatonin per gram of the mixture. And in a specific embodiment, the resulting thin-film mixture has a melatonin concentration of about 1.82 mg of melatonin per gram of the mixture. Moreover, according to these parameters, a milliliter of the thin-film mixture includes 1.82 mg of melatonin.

Block 320 of the method 300, in some embodiments, includes mixing additives with thin-film mixture to improve the structure of the resulting strip 100. For example, the thin-film mixture may further include oat fiber and/or hydrolyzed guar gum (i.e., Sunfiber®) to improve the structure of the resulting strip 100. Additionally or alternatively, the plasticizer may be added to the thin-film mixture to improve the flexibility and to reduce the stickiness of the strip 100.

A machine deposits the thin-film mixture onto a base or polished steel belt to form a thin film that is about 0.1 mm thick. The deposited thin film mixture is dried, resulting in a moisture loss (i.e., water loss) of from 85% to 99%. The water loss is from at least the water added to the liposomal base. The dried thin film is cut into pieces, which correspond to the dissolvable strip 100. Preferably, a moisture loss of 95% occurs from the thin-film mixture to the dissolvable strip 100. In an embodiment, the finished dissolvable strip 100 includes approximately 37 mg of melatonin per gram of dissolvable strip 100, such that a dissolvable strip 100 weighing 90 mg delivers a dosage of 3.3 mg of melatonin. The dissolvable strips 100 are packaged for sale.

Predetermined dosages of melatonin are possible by selecting a corresponding size of the dissolvable strip 100. Since, the plurality of liposomes 116 are substantially uniformly distributed throughout the base material 108, the dosage of melatonin in the strip 100 is easily controllable based the weight of the strip 100, the size of the strip 100, and/or the area of the strip 100. For example, doubling the area of the strip 100 is effective to double the dose of melatonin, and halving the area of the strip 100 is effective to half the dose of melatonin. Accordingly, the orally-dissolvable strip 100 includes a predetermined amount of the melatonin per unit of area of the strip 100. In an example, the predetermined amount provides about 1.0 mg of melatonin per square centimeter of the strip 100. In other embodiments, the predetermined amount provides about 0.25 mg to about 3 mg of melatonin per square centimeter of the strip 100.

The strip 100 provides the predetermined dosage of solubilized melatonin to the patient. Solubilized melatonin (as is encapsulated by the lipid shells 124 of the liposomes 116) is more effectively absorbed by the body as compared to crystalized or powdered melatonin (i.e., non-solubilized melatonin). Melatonin is difficult to solubilize in aqueous solution as exists in the mouth and digestive tract, because melatonin is only slightly soluble in water. It is estimated that only about 15% of non-solubilized melatonin taken orally in pill form or capsule form is actually absorbed into the body due to the low level of solubility of melatonin in water. Moreover, oral consumption of non-solubilized melatonin directs the melatonin into the digestive system where it is absorbed through the intestinal wall, into the bloodstream and transported to the liver and kidneys. The liver and kidneys remove 30-50% of melatonin from the bloodstream via CYP1A2 mediated 6-hydroxylation. Thus, it is advantageous to solubilize the melatonin prior to dosing and/or delivery. Solubilization of the melatonin increases the amount absorbed into the bloodstream by a factor of about 10 to 15 times, which allows less melatonin to be dosed, thereby reducing manufacturing and consumer cost. Stated differently, the solubilized melatonin of the dissolvable strip 100 when taken orally is 10 to 15 times more active and effective than non-solubilized melatonin taken orally.

The dissolvable strip 100, which is also referred herein as an ingestible therapeutic dosage form, is a more efficient melatonin delivery means than non-solubilized melatonin pills. The dissolvable strip 100 is configured to be placed into the mouth sublingually, under the tongue, allowing the melatonin to be absorbed directly into the bloodstream, avoiding a first pass through the liver. Specifically, in use, the base material 108 dissolves when in contact with the salvia and the digestive fluids in the mouth, thereby exposing the shells 124 of the liposomes 116 to the saliva and the digestive fluids. When the shells 124 of the liposomes 116 contact the salvia and the digestive fluids, the shells 124 dissolve and the melatonin solution 130 having the solubilized melatonin is exposed to the tissue of the patient's mouth for buccal and/or sublingual administration. The buccal and/or sub-lingual administration of fully-solubilized melatonin, makes available higher levels of melatonin to enter the bloodstream quickly as compared to administrating non-solubilized (i.e., crystalized and/or powdered) melatonin orally, buccally, or sublingually. The strip 100 is convenient, portable, stable, lightweight and easy to consume. Other solubilized melatonin products are dosed as capsules or liquids, which are less convenient, more difficult to swallow, and direct the product via the digestive system into the liver, where much of the melatonin is removed before reaching other cells of the body.

The strip 100 is effective for delivering melatonin for any known usage including as a sleep aid and as an ultraviolet (“UV”) light protectant (i.e., a sunscreen). An effective dose of solubilized melatonin for the induction of sleep is 2 mg-4 mg. The strip 100 provides such a dose in an easy to administer form, particularly for the many people that have trouble swallowing pills. With regard to UV protection, it is known that skin cells include receptor sites for melatonin that bind to melatonin, where the melatonin is available to neutralize free radical molecules created by exposure to UV radiation from the sun, in order to prevent sunburn, skin damage, and inflammation. Moreover, melatonin is more effective than sun blocking agents in preventing sunburn as it can be taken systemically (i.e., internally), using the strip 100, therefore, effectively protecting the entire body from UV radiation, not only the parts that have been covered by clothing or a sun blocking agent.

In another embodiment, any other supplement and/or compound that is soluble in glycerin is also included in the melatonin solution 130. Including melatonin as the supplement dissolved in the glycerin is only exemplary, and some other embodiments of the strip 100 do not include melatonin, but instead include at least one other glycerin-soluble supplement.

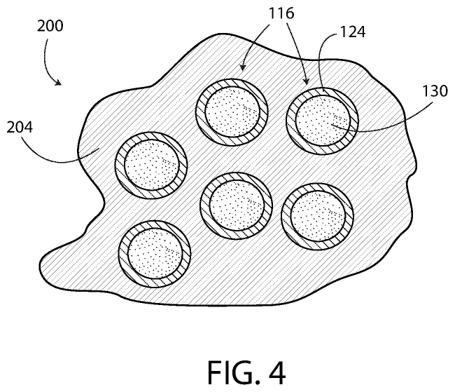

As shown in FIG. 4, a topical skin care product 200 includes the liposomes 116 having the melatonin solution 130 in a lotion base 204 instead the base material 108 of the dissolvable strip 100. The topical skin care product 200 includes, but is not limited to, a hand lotion, a hand cream, a skin lotion, a skin cream, a body moisturizer, and/or a skin moisturizer. Moreover, the skin care product 200 may be provided as a cream, a spray, and/or a lotion.

The lotion base 204 includes at least one of a skin moisturizer and a skin toner. The skin moisturizer is configured to increase water content in the external layers of the skin. An exemplary skin moisturizer includes glycerin, shea butter, and/or petrolatum. The skin toner is configured to clean the skin surface, to remove dead skin cells, and/or to exfoliate the skin. An exemplary skin toner includes salicylic acid, a calendula solution, and/or a chamomile solution. The lotion base 204 may include only the skin moisturizer, only the skin toner, or the skin moisturizer and the skin toner.

The lotion base 204 is compatible with the liposomes 116 and is mixed with the liposomes 116. In storage, the components of the skin moisturizer and/or the skin toner do not react with or degrade the lipid shells 124, such that the liposomes 116 are stable in the skin care product 200. As used herein, the liposomes 116 are stable, when the liposomes 116 do not dissolve, deteriorate, break, and/or rupture during storage of the skin care product 200. In addition to protecting and/or cleaning the skin, the lotion base 204 is a carrier for the liposomes 116.

The liposomes 116 of the skin care product 200 are the same liposomes 116 as discussed above in connection with the dissolvable strip 100. Accordingly, the liposomes 116 include the melatonin mixture 130 that includes solubilized melatonin in liquid glycerin. Moreover, the lipid shells 124 of the liposomes 116 of the skin care product 200 prevent crystallization of the melatonin, such that the melatonin of the melatonin solution 130 remains solubilized in the glycerin within the liposomes 116.

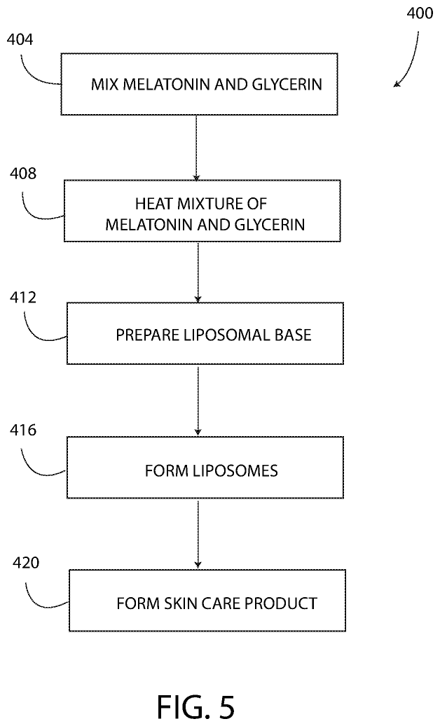

As shown in FIG. 5, the skin care product 200 is made according to an exemplary method 400. As shown in block 404, first the method 400 includes mixing melatonin with liquid glycerin. The melatonin may be dry and/or powered, and is typically in a crystalized state. As noted above, in an exemplary embodiment, at least 250 mg of melatonin is added per milliliter of glycerin. When the melatonin is added to room temperature (i.e., 25° C.) glycerin, only a portion of the melatonin dissolves in the glycerin.

Next, at block 408 of the method 400, the melatonin and glycerin mixture is heated to approximately 76.6° C. (170° F.). Moreover, in some embodiments, the melatonin and glycerin mixture is stirred during the heating process. As the mixture is heated and stirred, all of the melatonin dissolves in the glycerin to form the melatonin solution 130.

At block 412 of the method 400, the liposomal base is produced with a mixture of water and purified soy phosphatidylcholine. In an exemplary embodiment, about 40 parts water are combined with 1 part soy phosphatidylcholine to form the liposomal base. The mixture is heated to 48.9° C. (120° F.) while being stirred to complete the liposomal base. In another exemplary embodiment, about 70-75 parts water are combined with 5 to 15 parts soy phosphatidylcholine to form the liposomal base.

At block 416, the liposomes 116 are formed. First, the melatonin solution 130 is combined with the liposomal base. In an exemplary embodiment, about 3 parts liposomal base is combined with 1 part of the melatonin solution 130. Accordingly, the combined ingredients as a percentage by weight include 70% to 75% water, 5% to 15% soy phosphatidylcholine, 0.5% to 3% glycerin, and 0.25% to 1% melatonin.

Next, at block 416 of the method 400, the mixture of the melatonin solution 130 and the liposomal base is sonicated while being further mixed together to form the liposomes 116. The sonication results in the liposomal base forming the lipid shells 124 around very small portions of the melatonin solution 130. Any other suitable process may be used to form the liposomes 116 including, but not limited to, high-shear mixing, extrusion, micromixing, and the Mozafari method. Moreover, in one embodiment, after the liposomes 116 are formed, the mixture of the melatonin solution 130 and the liposomal base is sonicated without mixing for an additional fifteen to thirty seconds. The concentration of melatonin in the liposomes 116 is from about 3 mg/ml to about 7 mg/ml. Preferably, the concentration of melatonin in the liposomes 116 is about 5 mg/ml. For example, the mixture of the melatonin solution 130 and the liposomal base forms a fluid mixture that is the plurality of liposomes 116 and that can be measured by volume. A milliliter of the plurality of liposomes 116 includes, for example, 5 mg of melatonin.

In one embodiment, the melatonin solution 130 is maintained at a temperature of 76.6° C. until it is combined with the liposomal base. The combination of the melatonin solution 130 and the liposomal base is mixed and sonicated quickly (i.e., within five minutes and preferably within two minutes) of being combined to prevent the temperature of the melatonin solution 130 from dropping to an unsuitable level. By maintaining the temperature of the melatonin solution 130 at or near 76.6° C. prior to combining with the liposomal base, the melatonin stays fully solubilized within the melatonin solution 130 without settling, precipitating, crystalizing, or recrystallizing. In one embodiment, the liposomes 116 are stable for a few days (i.e., three days) prior to being mixed with the base material 108. Preferably, however, the liposomes 116 are incorporated into the base material 108 shortly after production by vigorous mixing that does not damage or break the liposomes 116.

At block 420, the liposomes 116 are combined and mixed with the lotion base 204. In particular, a predetermined amount of the liposomes 116 (measured by volume or weight) is added to a predetermined amount of the lotion base 204. In an exemplary embodiment, the liposomes 116 are mixed with the lotion base 204 at ratio of one part liposomes 116 to nine parts lotion base 204. The melatonin of the melatonin solution 130 is prevented from crystalizing in the skin care product 200 by the lipid shell 124 of the liposomes 116. In one embodiment, the concentration of melatonin in the skin care product 200 is from 0.25% to 1% w/w. In a preferred embodiment, the concentration of melatonin in the skin care product is about 0.5% w/w.

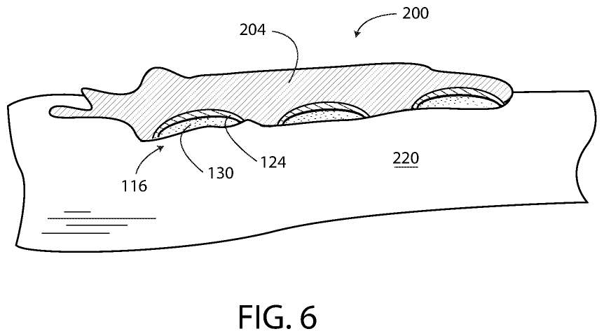

With reference to FIG. 6, in use, the skin care product 200 including the liposomes 116, is topically applied to the skin 220 of a patient. In FIG. 6, the liposomes 116 are shown in a very much enlarged state for ease of illustration and explanation. The applied skin care product 200 is gently spread, rubbed, or massaged over the area of the skin 220 requiring treatment. As the skin care product 200 is applied to the skin, the spreading breaks and/or ruptures the liposomes 116, such that the melatonin solution 130 is applied directly to the skin 220. The liposomes 116 easily break in response to the spreading and the user does not need to forcibly try to break the liposomes 116. Simply, comfortably spreading the skin care product 200 onto the skin in the typical manner is sufficient for breaking the liposomes 116.

As shown in FIG. 6, each of the three liposomes 116 has a broken lipid shell 124. When the lipid shell 124 is broken, the melatonin solution 130 having the solubilized melatonin escapes the encapsulation of the liposome 116 and the solubilized melatonin contacts directly the skin 220 of the patient. The glycerin of the melatonin solution 130 also contacts directly the skin 220 and further moisturizes and protects the skin 220.

The skin care product 200 including the liposomes 116 encapsulating the solubilized melatonin, provides numerous benefits when topically applied to the skin 220 of the patient including protection from UV radiation damage as occurs in sunburn. Moreover, the skin care product 200 is useful for the treatment of persistent skin rashes, rosacea, gout, insect bites, poison ivy, varicose veins, and/or joint inflammation due to arthritis. All of these conditions, and others, involve localized inflammation of the affected tissue which causes pain, swelling, itching and restriction of motion. Itching and scratching of the problem area causes damage, resulting in a negative spiral of elevated immune response and further tissue damage. The topically applied solubilized melatonin from the skin care product 200 reduces the inflammation and calms the immune response, thereby stopping the urge to itch and breaking the spiral, allowing the body to heal itself. Moreover, it has been found that a topical application of the solubilized melatonin, as included in the skin care product 200 having the liposomes 116, directly to the site requiring treatment is far more effective than oral administration of melatonin. Beneficial results are observed in hours from the skin care product.

In addition to the above, the melatonin solution 130 is a novel treatment for severe lung trauma. Many people suffer from severe lung trauma, which may occur as a result of viral infection (such as the novel coronavirus (SARS-CoV-2 virus, COVID-19)), asthma, chronic obstructive pulmonary disease (“COPD”), lung irritation due to air pollution and/or smoking, other inflammatory conditions of the lungs, and other causes. For example, a viral infection or insult to a patent's lung tissue causes some patients to exhibit an over-reaction of the immune system known as hypercytokinemia or colloquially as a “cytokine storm.” Hypercytokinemia is a severe immune reaction in which the body releases too many cytokines into the bloodstream too quickly. While cytokines are part of a healthy immune response, an abundance of cytokines harms the body and may result in fever, inflammation, fatigue, and nausea among other negative responses. The cytokine storm then builds upon itself in a positive feedback loop, because these negative responses result in the release of additional cytokines, which may cause further severe damage to healthy lung tissue, fluid accumulation in the alveoli, loss of lung function, and in severe cases death. Moreover, these negative responses to the abundance of cytokines also result in the production of many free radical molecules, which of course are also harmful to the patient. Problematically, there are not always adequate treatments available for severe lung trauma and hypercytokinemia. Moreover, at times, the treatment for severe lung trauma is primary supportive with only supplemental oxygen and, for the most traumatized patients, mechanical ventilation, an undesirable option for most patients.

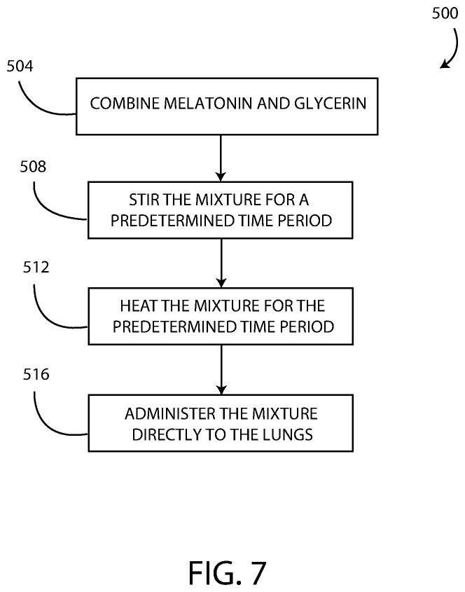

As shown in FIG. 7, a method 500 is disclosed herein that delivers melatonin molecules in the form of the melatonin solution 130 directly into a patent's lungs to mitigate the patient's negative response(s) to viral infections, lung tissue trauma, and hypercytokinemia by at least reducing inflammation, removing free radicals, and soothing the patient's airways. As such, the melatonin solution 130 reduces, prevents, and/or treats lung damage and other damage as a result of viral infection and/or hypercytokinemia.

At block 504 of the method 500, the melatonin is combined with glycerin in a glass vessel (not shown) or any other heat-safe and non-reactive container. The melatonin is added to the glycerin in any desired form including crystalline and pulverized.

Next, at blocks 508 and 512 of the method 500, the melatonin and glycerin mixture is stirred and heated for a predetermined time period. An exemplary predetermined time period is from two to ten minutes. In one embodiment, the predetermined time period is about five minutes. The melatonin and glycerin mixture is heated to approximately 76.6° C. (170° F.) during the predetermined time period.

The stirring and heating of blocks 508 and 512 dissolves the melatonin into the glycerin to form the melatonin solution 130. As noted above, the melatonin solution 130 is a homogenous liquid including molecules of melatonin that are fully dissolved into the liquid glycerin with no clumps, crystals, or other solid particles of melatonin remaining.

The melatonin mixture 130 is then cooled to room temperature. The melatonin mixture 130 is stable and no settling or precipitating of the melatonin occurs in the glycerin even after six weeks of sitting at room temperature.

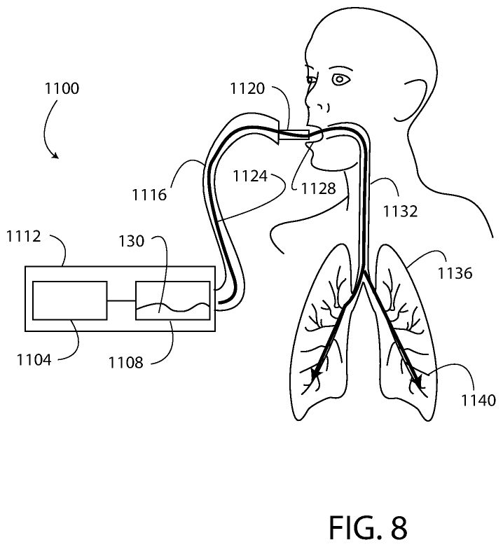

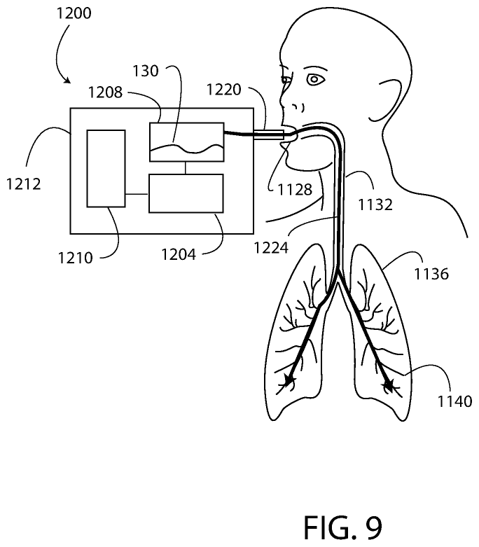

Next, at block 516 of the method 500 the melatonin mixture 130 is administered directly to the lungs of a patient or animal via at least one form of inhalation administration or inhalation therapy including nebulization (FIG. 8) and vaporization (FIG. 9). As illustrated herein and as described below, inhalation administration enables the melatonin of the melatonin mixture 130 to pass into a patient's mouth or nose, through the trachea, and directly into the patient's lungs through the left and right bronchus. From the bronchus, the inhaled melatonin mixture 130 passes into the bronchioles and makes direct contact with the alveoli. Thus, the inhaled melatonin mixture 130 contacts the patient's entire airway and is available to contact directly and to treat the entire airway including the trachea, the left and right bronchus, the bronchioles, and the alveoli. Moreover, the inhaled melatonin mixture 130 passes to the patient's bloodstream due to the direct contact of the melatonin with the alveoli. As a result, the inhaled melatonin mixture 130 is also carried throughout the patient's whole body via blood flow. This makes the inhaled melatonin mixture 130 available to remove free radicals, treat inflammation, and provide numerous other health benefits to substantially all cells in the patient's body. As a powerful antioxidant, melatonin administered directly to the lung tissue sufficiently moderates free radical production by the immune system to reduce or eliminate damage to healthy lung tissue, reduce inflammation and accumulation of fluid in the alveoli, maintain effective lung function, allow the damaged tissue to heal, while permitting the immune system to effectively destroy viral pathogens, for example.

With reference to FIG. 8, a nebulizer system 1100 is shown and nebulization is the form of inhalation administration. The nebulizer system 1100 includes a nebulizer unit 1104 operably connected to a reservoir 1108. The nebulizer unit 1104 and the reservoir 1108 are located within a housing 1112. Flexible tubing 1116 is operably connected to the nebulizer unit 1104, the reservoir 1108, and/or the housing 1112. The tubing 1116 is terminated in a mouthpiece 1120. The nebulizer system 1100 is shown as a stationary device, but in other embodiments the nebulizer system 1100 is provided as a handheld system or any other suitable system for nebulization of a liquid.

The nebulizer unit 1104 is configured to nebulize the liquid contents of the reservoir 1108 in order to form a mist and/or an aerosol 1124 that travels through the tubing 1116 to the mouthpiece 1120. The nebulizer unit 1104 is provided as a jet nebulizer unit, an ultrasonic nebulizer unit, and/or a mesh nebulizer unit. The nebulizer unit 1104 is supplied with electrical energy from a suitable power source such as a wall outlet or a battery (not shown).

The patient places the mouthpiece 1120 in his mouth and inhales through the mouthpiece 1120. When the patient inhales, the aerosol 1124 is drawn out of the housing 1112, through the tubing 1116, out of the mouthpiece 1120, into the patient's mouth 1128, through the patient's trachea 1132, and into the patient's lungs 1136. The nebulizer unit 1104 is configured to form a fine aerosol without large droplets, so that the aerosol 1124 is drawn deep into the lungs 1136 and in contact with the alveoli 1140.

According to block 516 of the method 500 of FIG. 7, the melatonin mixture 130 is added to the reservoir 1108 and then the nebulizer unit 1104 is activated. The nebulizer unit 1104 generates the aerosol 1124 directly from the dissolved melatonin mixture 130, such that the melatonin mixture 130 is inhaled deep into the patient's lungs 1136 through tubing 1116 and the mouthpiece 1120. In one embodiment, the nebulization unit 1104 is configured to nebulize the melatonin mixture 130 into the aerosol 1124 having liquid particles between 0.1 and 10 μm in diameter that are suitable for breathing and administering the melatonin directly into the lungs 1136. The tubing 1116 may also be connected to an oxygen mask (not shown), a ventilator tube of a mechanical ventilator (not shown), or an intubation tube (not shown) as an alternative to supplying the aerosol 1124 to the patient's lungs 1136 through the mouthpiece 1120.

In one embodiment, only the melatonin mixture 130 is added directly to the reservoir 1108 with no other liquid inhalation agents. In another embodiment, the melatonin mixture 130 is combined with a liquid inhalation agent, such as a saline solution or any other suitable liquid for the nebulization process, to form an inhalation mixture. The inhalation mixture is added to the reservoir 1108, and the aerosol 1124 is generated from the inhalation mixture. In each embodiment, the dissolved melatonin mixture 130 provides a convenient means of dosing a predetermined amount of melatonin by easily measuring a liquid quantity of the melatonin mixture 130. Accordingly, the melatonin mixture 130 is easily dosed into a quantity of saline solution or other solution.

In a further embodiment, an aqueous liposomal dispersion and/or liposomal matrix including the liposomes 116 of FIG. 2 and an inhalation agent (not shown) are added to the reservoir 1108 of the nebulizer system 1100 to form an inhalation mixture. Suitable inhalation agents include saline solution and any other suitable liquid. The nebulization unit 1104 is configured to nebulize the inhalation mixture into the aerosol 1124 having liquid particles between 0.1 and 10 μm in diameter that are suitable for breathing and delivering the liposomes 116 directly into the lungs 1136. Accordingly, each particle includes a plurality of liposomes 116. Nebulization in this manner has been shown to be effective in delivering the liposomes 116 deep into the lungs 1136 including reaching the alveoli 1140. The inhalation mixture does not include any toxic or irritating components that could cause damage to the highly-sensitive lung tissue.

The aqueous, saline liposomal dispersion of melatonin (i.e. the inhalation mixture) contains the solubilized melatonin molecules at a concentration of about 1.0 mg per ml. Such a dosage is a safe and sufficient to mitigate excess free radical damage and reduce inflammation, without any negative side effects. Melatonin molecules from the liposomes 116 when in the body diffuse within minutes into cells and mitochondria to absorb excess free radical electrons where they are produced. Melatonin diffuses readily through cell walls. Melatonin has a short residence time in the body, with about 30%-50% of melatonin in the blood being removed during each pass through the liver. This makes it difficult to achieve adequate and sustained blood concentrations of melatonin and demonstrates the advantage of directly applying the solubilized melatonin of the melatonin mixture 130 to the lung tissue at an effective concentration, thereby avoiding hepatic removal of the melatonin (i.e., removal of the melatonin from the blood by the liver).

As shown in FIG. 9, a vaporizing system 1200 is shown and vaporization or “vaping” is the form of inhalation administration. The vaporizing system 1200 includes a heating element 1204 operably connected to a reservoir 1208 and a power source 1210, such as a rechargeable battery. The heating element 1204, the reservoir 1208, and power source 1210 are located within a housing 1212 (not shown to scale). A mouthpiece 1220 extends from the housing 1212.

The heating element 1204 is provided as a resistive heating element, for example, and is configured to vaporize the liquid contents of the reservoir 1208 in order to generate a vapor 1224 that is drawn out of the mouthpiece 1220 by the patient. The heating element 1204 vaporizes the liquid contents of the reservoir 1208 without combusting or burning the liquid contents of the reservoir 1208. In particular, the patient places the mouthpiece 1220 in his mouth 1128 and inhales through the mouthpiece 1220. When the patient inhales, the vapor 1224 is drawn out of the mouthpiece 1220, into the patient's mouth 1128, through the patient's trachea 1132, and into the patient's lungs 1136. The vapor 1224 includes very small particles that are drawn deep into the lungs 1136 and in contact with the alveoli 1140.

With reference again to block 516 of the method 500 of FIG. 7, the melatonin mixture 130 is added to the reservoir 1208 and then the heating element 1204 is activated. The heating element 1204 vaporizes the melatonin mixture 130 to form the vapor 1224. The vapor 1224 includes molecules of melatonin.

In one embodiment, only the melatonin mixture 130 is added directly to the reservoir 1208 with no other liquid vaporization agents. In another embodiment, the dissolved melatonin mixture 130 is combined with a liquid vaporization agent such as propylene glycol or any other suitable liquid for the vaporization process, to form a vaporization mixture. The vaporization mixture is then added to the reservoir 1208. The melatonin mixture 130 provides a convenient means of dosing a predetermined amount of melatonin by easily measuring a liquid quantity of the melatonin mixture 130. Accordingly, the melatonin mixture 130 is easily dosed into a quantity of suitable vaporization agent.

Moreover, the vaporizing system 1200 is compatible with an aqueous liposomal dispersion and/or liposomal matrix including the liposomes 116 of FIG. 2 and an inhalation agent. Again, suitable inhalation agents include propylene glycol and the like. The heating element 1204 is configured to vaporize the aqueous liposomal dispersion into the vapor 1224 that includes the melatonin molecules and the liposomes 116. Vaporization in this manner has been shown to be effective in delivering the melatonin molecules and the liposomes 116 of the vapor 1224 deep into the lungs 1136 including reaching the alveoli 1140. The aqueous liposomal dispersion does not include any toxic or irritating components that could cause damage to the highly-sensitive lung tissue.

While the disclosure has been illustrated and described in detail in the drawings and foregoing description, the same should be considered as illustrative and not restrictive in character. It is understood that only the preferred embodiments have been presented and that all changes, modifications and further applications that come within the spirit of the disclosure are desired to be protected.

What is claimed is:

1. A method of making an orally-dissolvable strip, comprising:
combining melatonin with glycerin;
forming a melatonin solution by heating the combined melatonin and glycerin to dissolve the melatonin in the glycerin;
forming a plurality of liposomes by mixing a liposomal base with the melatonin solution, each liposome of the plurality of liposomes including a lipid shell of the liposomal base encapsulating a portion of the melatonin solution; and
forming the orally-dissolvable strip by combining the plurality of liposomes with a base material,
wherein the base material and the liposomal base are orally-dissolvable, and
wherein the lipid shell prevents crystallization of the dissolved melatonin in the glycerin, such that within each liposome of the plurality of liposomes the melatonin is dissolved in the glycerin.

2. The method as claimed in claim 1, wherein the liposomes of the plurality of liposomes are substantially uniformly dispersed throughout the base material.

3. The method as claimed in claim 2, wherein the orally-dissolvable strip has a predetermined amount of the melatonin per unit of area of the orally-dissolvable strip.

4. The method as claimed in claim 1, wherein:
the orally-dissolvable strip is an ingestible therapeutic dosage form, and
the liposomes of the plurality of liposomes are dispersed homogenously throughout the orally-dissolvable strip.

5. The method as claimed in claim 1, wherein the base material is edible and includes a polysaccharide polymer.

6. The method as claimed in claim 1, wherein combining the melatonin with the glycerin comprises:
combining crystalized melatonin with liquid glycerin.

7. The method as claimed in claim 6, wherein heating and mixing the melatonin and the glycerin comprises:
heating the melatonin and the glycerin to 170° F. to dissolve the crystalized melatonin into the liquid glycerin.

8. The method as claimed in claim 1, wherein forming the orally-dissolvable strip comprises:
mixing the plurality of liposomes with the base material without breaking the lipid shells of the plurality of liposomes, such that the lipid shells encapsulate the melatonin solution when the plurality of liposomes are formed into the orally-dissolvable strip.

9. An orally-dissolvable strip, comprising:
an orally-dissolvable base material;
a melatonin solution including solubilized melatonin in liquid glycerin; and
a plurality of liposomes fixedly positioned throughout the base material, each liposome of the plurality of liposomes having a lipid shell encapsulating a portion of the melatonin solution,
wherein the lipid shell is formed from an orally-dissolvable liposomal base, and
wherein the lipid shell is configured to prevent crystallization of the melatonin of the encapsulated portion of the melatonin solution, such that each liposome of the plurality of liposomes contains the solubilized melatonin in liquid glycerin.

10. The orally-dissolvable strip as claimed in claim 9, wherein:
the liposomes of the plurality of liposomes are substantially uniformly dispersed throughout the orally-dissolvable base material, and
the orally-dissolvable strip is an ingestible therapeutic dosage form.

11. The orally-dissolvable strip as claimed in claim 10, wherein the orally-dissolvable strip has a predetermined amount of the melatonin per unit of area of the orally-dissolvable strip.

12. The orally-dissolvable strip as claimed in claim 9, wherein the base material is edible and includes a polysaccharide polymer.

13. The orally-dissolvable strip as claimed in claim 9, wherein a thickness of the orally-dissolvable base material is less than one millimeter.

14. The orally-dissolvable strip as claimed in claim 9, wherein the lipid shell includes at least one lipid bilayer.

15. The orally-dissolvable strip as claimed in claim 14, wherein the lipid shell is formed from phosphatidylcholine.

16. A topical skin care product, comprising:
a lotion base;
a melatonin solution including solubilized melatonin in liquid glycerin; and
a plurality of liposomes mixed with the lotion base, each liposome of the plurality of liposomes having a lipid shell encapsulating a portion of the melatonin solution,
wherein the lipid shell is configured to prevent crystallization of the melatonin of the encapsulated portion of the melatonin solution, such that each liposome of the plurality of liposomes contains the solubilized melatonin in liquid glycerin.

17. The topical skin care product as claimed in claim 16, wherein the liposomes of the plurality of liposomes are configured to break upon application of the topical skin care product to skin to release the melatonin solution onto the skin.

18. The topical skin care product as claimed in claim 16, wherein the lipid shell includes at least one lipid bilayer.

19. The topical skin care product as claimed in claim 18, wherein the lipid shell is formed from phosphatidylcholine.

20. The topical skin care product as claimed in claim 16, wherein:
the lotion base includes at least one of a skin moisturizer and a skin toner, and
the lotion base is compatible with the plurality of liposomes, such that the plurality of liposomes are stable.
